(19) United States
(12) Patent Application Publication
(10) Pub. No.: US 2022/0032011 A1
(43) Pub. Date: Feb. 3, 2022

(54) Microminiature EM Coil Sensor Pull Ring For Catheter

(71) Applicant: IntriCon Corporation, Arden Hills, MN (US)

(72) Inventors: Daniel J. Potter, Stillwater, MN (US); Alexander L. Darbut, Edina, MN (US); David Bosch, Circle Pines, MN (US); Sam A. Puent, Minneapolis, MN (US)

(21) Appl. No.: 17/388,119

(22) Filed: Jul. 29, 2021

Related U.S. Application Data
(60) Provisional application No. 63/058,380, filed on Jul. 29, 2020.

(51) Int. Cl. A61M 25/01

(52) U.S. Cl. CPC A61M 25/0158; A61M 25/0147; A61M 25/0138; A61M 2025/0166; A61M 2207/00; A61M 2205/0244; A61M 2205/0272; A61M 2205/3317; A61M 2025/0163

(57) ABSTRACT

A microminiature electro-magnetic coil sensor pull ring with a pull wire attached thereto is used in changing the angle of a distal end of a medical catheter. The tubular pull ring has a connection recess with a flat bottom machined into the full wall thickness and located proximal to a coil wrap area. Circuit wires are electrically connected to the two lead ends of the coil within the connection recess, such that neither the circuit wires nor the lead ends stand proud of the full wall thickness. The coil wrap area is also recessed, and can have side walls defining an offset angle for the turns of the coil. In another aspect, a coil is wound around one of the pull wires for the pull ring.

### CROSS-REFERENCE TO RELATED APPLICATION(S)

The present application claims the benefit of U.S. provisional patent application Ser. No. 63/058,380, filed Jul. 29, 2020, entitled “Embedded EM Sensors Integrated Into Surgical Navigational Catheters And Diagnostic Devices”. The contents of U.S. provisional patent application Ser. No. 63/058,380 are hereby incorporated by reference in entirety.

### BACKGROUND OF THE INVENTION

Microminiature electrical coils are used in various types of electronic and medical equipment, with an example being the AURORA electromagnetic tracking system provided by Northern Digital Inc. d/b/a NDI. Such electromagnetic tracking systems utilize a sensor coil to read and/or respond to electromagnetic fields, with a microprocessor based system interpreting the electrical or magnetic response to determine a location of the coil in three-dimensional space. U.S. Pat. Nos. 6,288,785, 6,385,482, 6,553,326, 6,625,465, 6,836,745, 7,353,125, 7,469,187, 7,783,441 and 7,957,925 describe such systems, incorporated by reference.

A preferred prior art coil used in the electromagnetic tracking system uses an extremely thin copper wire (such as 58 American Wire Gauge (AWG), i.e., 0.00039″ in diameter) wound around a core. The core may be a solid cylinder or a hollow tube or lumen. The core is typically formed of a ferrite-based or soft magnetic material, with a preferred core material being mu-metal. The core may be coated with a parylene layer to provide insulation. The electro-magnetic (EM) sensor coil is typically quite small, and is placed in the catheter shaft wall or interior of catheter. An application of such systems is with the coil configured as part of a catheter, to electromagnetically track the location of the catheter coil within the human body during a medical procedure. For instance, example applications include the use of the sensor coil in pulmonary bronchoscopy, mapping catheters, ablation catheters, diagnostic catheters and electrophysiology (EP) catheters.

In the prior art manufacturing assembly process for creating the EM sensor coil, two wires are used as leads for the coil, with the two leadwires being twisted into a twisted pair. The leadwires are typically thicker than the coil wire, such as 40 AWG (i.e., 0.003145″, or about eight times the diameter of the coil wire) leadwires encased in insulation but with their ends stripped. Since the coil wire is very tiny, it is difficult to attach the larger 40 AWG lead wires to the smaller 58 AWG coil wire ends. The typical connection between the coil wire and the leadwires involves crudely wrapping the coil wire ends around each leadwire end and then soldering. The sensor coil is encapsulated, such as with a biocompatible ultra-violet adhesive over the top of the coil windings, termination points, and a minimum of three twists of sensor leadwires.

Prior art EM sensor coils are typically somewhat small and fragile, and problems can occur with prior art EM sensor coils when being handled assembled into the catheter structure. One or both of the flexible ends of the coil wires may break, as well as one or both leadwires, or one or both ends of the coil wire, pulling out of the adhesive encapsulation. Additionally, because the EM sensor coil diameter is generally somewhat smaller than the diameter of the catheter it is a component of, a location offset can be introduced with the EM sensor coil axis being different from (and possibly skewed relative to) the catheter axis.

Separately, pull ring assemblies can be utilized in medical catheters to provide catheter steering capabilities. A pull ring with steering wire assembly can incorporate a single pull-wire attached to the pull ring or a plurality of pull wires attached to the pull ring to accommodate bi-directional or multi-directional steering. An example of a 0.1″ diameter stainless steel pull ring using two 0.004×0.012″ flat (equivalent to about 32 AWG) stainless steel pull wires is disclosed in U.S. Pat. Pub. No. 2007/0299424, incorporated by reference.

These various prior art structures have their own cost and space requirements and introduce potential failure locations into the final catheter product. Better solutions are needed.

### BRIEF SUMMARY OF THE INVENTION

The present invention is a microminiature electro-magnetic coil sensor pull ring for use in changing the angle of a distal end of a medical catheter for navigation of the catheter through human tissue, and a method of manufacturing such a microminiature electro-magnetic coil sensor pull ring. The one aspect, the tubular pull ring has geometric features which facilitate having the coil formed about the tubular pull ring. One such geometric feature is a connection recess into the full wall thickness and located proximal to the coil wrap area. Circuit wires are electrically connected to the two lead ends of the coil within the connection recess, such that neither the circuit wires nor the lead ends stand proud of the full wall thickness. In another aspect, a coil is wound around one of the pull wires for the pull ring, and electrical connections can still be made within one or more connection recesses of the pull ring.

### BRIEF DESCRIPTION OF THE DRAWINGS

FIG. 1 is a greatly enlarged perspective view, from the proximal end, of a first preferred embodiment of a microminiature EM coil sensor pull ring in accordance with the present invention.

FIG. 2 is a perspective view, from the distal end, of the preferred pull ring used in the microminiature EM coil sensor pull ring of FIG. 1.

FIG. 3 is a perspective view, slightly from the proximal end, of a second preferred pull ring, at a rotational position which shows the offset angle θ well.

FIG. 4 is a perspective view, slightly from the proximal end, of a third preferred pull ring, at a rotational position which is rotated about 135° clockwise relative to the rotational position shown in FIG. 3.

FIG. 5 is a perspective view, from the proximal end, of a fourth preferred embodiment of a microminiature EM coil sensor pull ring in accordance with the present invention.

While the above-identified drawing figures set forth preferred embodiments, other embodiments of the present invention are also contemplated, some of which are noted in the discussion. In all cases, this disclosure presents the illustrated embodiments of the present invention by way of representation and not limitation. Numerous other minor modifications and embodiments can be devised by those skilled in the art which fall within the scope and spirit of the principles of this invention.

### DETAILED DESCRIPTION

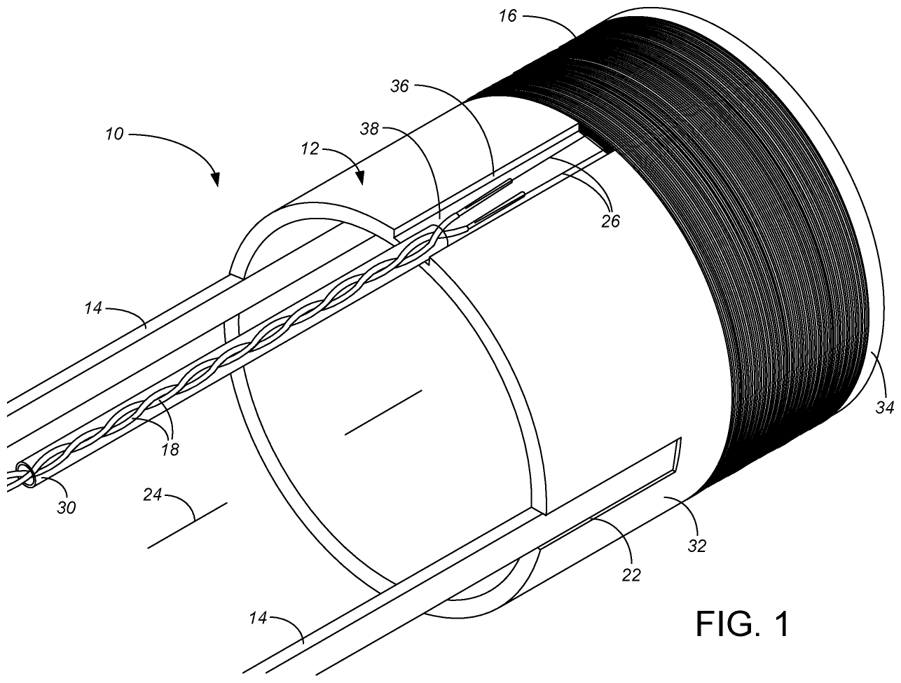

FIG. 1 shows a first preferred embodiment of a microminiature electrical coil sensor pull ring 10 of the present invention, intended for use as a component in a catheter assembly (not shown). The coil sensor pull ring 10 includes components which are primarily structural of a rigid tubular pull ring 12 guided by at least one but more commonly a plurality of pull wires 14. The coil sensor pull ring 10 includes components which are primarily electrical and/or magnetic of a wire coil 16 and electrical circuit wires 18 for the wire coil 16.

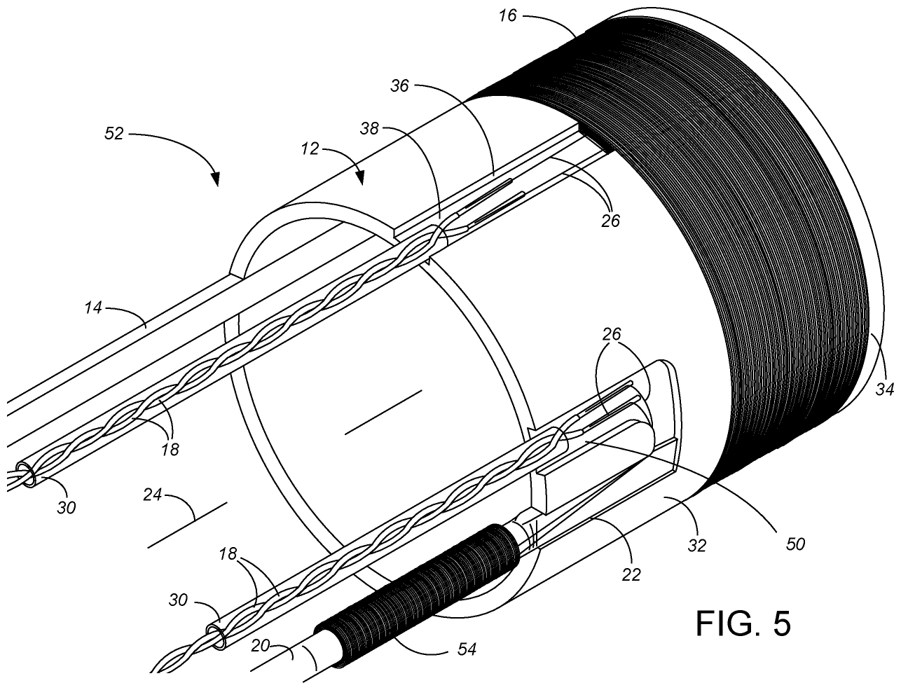

The pull ring 12 is placed in the distal end of the catheter adjacent the catheter tip, and is used to bend the distal end of the catheter during navigation through human tissue (such as an artery or vein) so the catheter can be advanced to the desired catheter deployment site. The pull wires 14 must have sufficient flexibility to curve through the catheter path within the human tissue, while being able to support the pull force needed to deflect the angle of the pull ring 12 for navigation as the catheter is advanced into the human body. In the first example shown, the pull wires 14 have a rectangular cross-section, oriented to match the primary navigational direction intended, i.e., as shown in the orientation of FIG. 1, the pull wires 14 are used to deflect the catheter tip in the left-to-right direction, and the pull wires 14 have a greater height than width so they are more flexible in the left-to-right direction than the up-and-down direction. The pull wire height should be less than 50% of the pull ring outer diameter, with FIG. 1 showing a pull wire 14 with a height which is about 8% of the pull ring outer diameter. Other embodiments utilize pull wires of different cross-sections for part or all of their length, such as the circular cross-sectioned pull wire 20 shown in FIG. 5.

In the preferred embodiments, the pull wires 14 are attached to the pull ring 12 by cutting a longitudinally-extending pull ring slot 22 in the proximal end of the pull ring 12 for each pull wire 14, and then laser welding the pull wire 14 to the pull ring 12 within the slot 22. The preferred laser welding positions the pull wire 14 within its slot 22 so the pull wire 14 does not stand proud or extend beyond either the inner diameter or the outer diameter of the tubular pull ring shape, which facilitates both assembly with other catheter structures and functionality of the pull ring 12 with minimal friction/opportunity for snagging during assembly and use of the catheter.

The pull ring 12 has an outer diameter which is a majority (i.e., at least 50%) of the outer diameter of the distal end of the catheter in which it is used, and more preferably from 80 to 100% of the outer diameter of the distal end of the catheter in which it is used. In most catheter assemblies, the pull ring axis 24 is coincident with the catheter axis (not separately shown). Catheter diameters depend heavily upon the particular intended deployment site, but pull ring outer diameters are typically in the range of 3 to 34 French (0.039-0.445″, or 1-11.3 mm).

The wall thickness of the pull ring 12 should be as thin as possible to provide as much interior space as possible, while still supporting the rigidity of the pull ring 12 in use. For pull rings formed of metal, the wall thickness will typically be 2-20% of the outer diameter, with the example shown having a wall thickness of about 5% of the outer diameter.

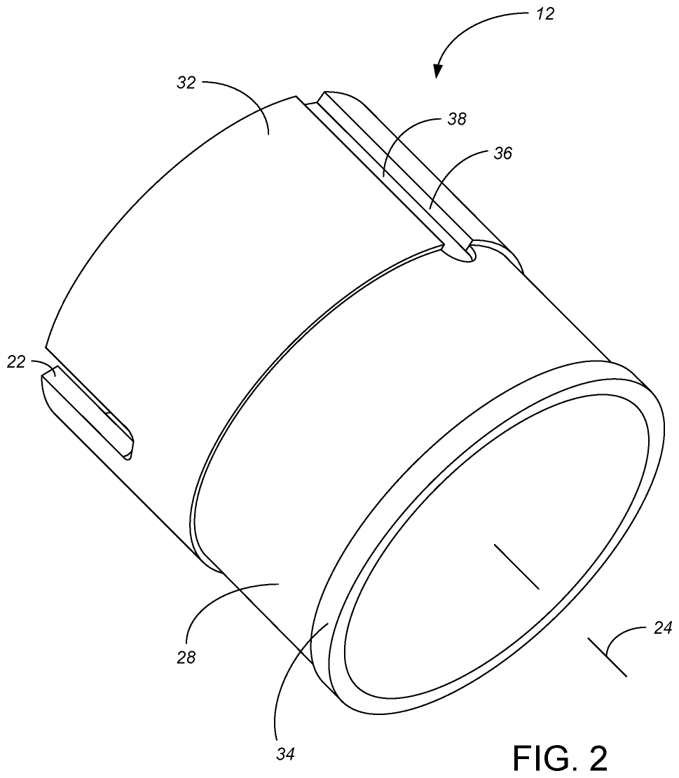

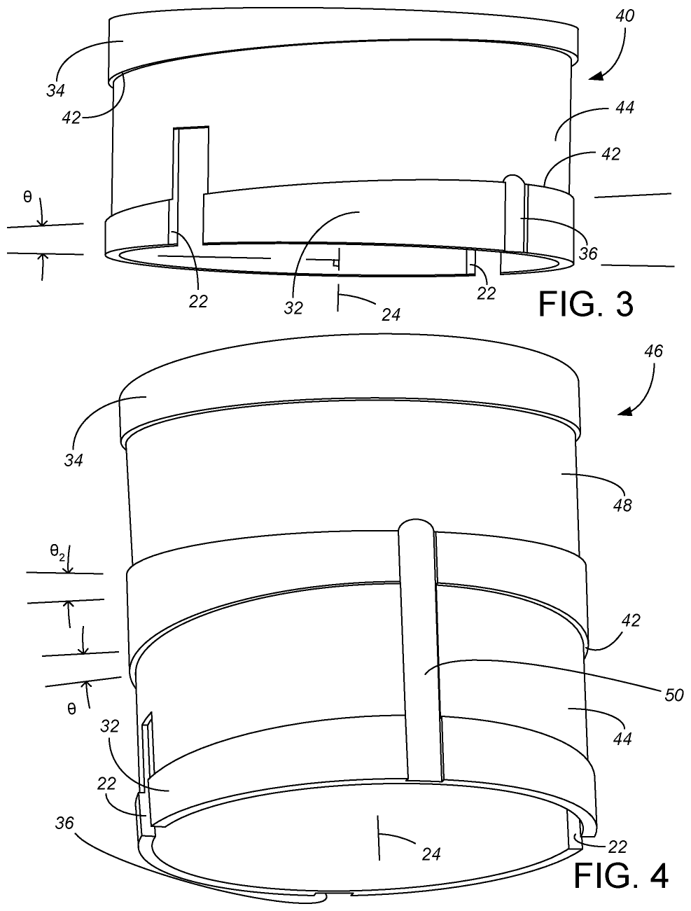

The pull ring 12 of the present invention has a length in the same order of magnitude as its outer diameter, such as within a range of 30-300% of the outer diameter, which will typically make the tubular pull ring 12 between 0.05″ and 0.5″ in length. The particular embodiments shown in FIGS. 1, 2 and 5 have a length which is about 5% longer than its diameter, but FIGS. 3 and 4 show both shorter and longer pull rings. The pull ring 12 is most preferably cylindrical, but could alternatively have a more square, rectangular or other polygon or oval shape.

The material chosen for the pull ring 12 is selected for sterility and as being biologically compatible for the likelihood of contact with body tissues for the length of time the catheter is within the body, and also for having both high magnetic permeability and high strength. The requirements for high magnetic permeability and high strength are important due to the interaction between the pull ring 12 and the primary electrical/magnetic components of the coil sensor pull ring 10. To maximize magnetic permeability, the material for the pull ring 12 should be a ferrite-based or soft magnetic material, with one preferred material being mu-metal. More preferably, a ferritic stainless steel in the full-hard condition is used for the pull ring material to better satisfy physical requirements. The preferred material choice is a 400-series ferritic stainless steel, which maximizes EM sensor sensitivity and is chemically suited for medical purposes. Most preferred materials include SS410 and SS 416, with a potential to use SS444 if extra corrosion-resistance is preferred. Catheter applications that are tolerant of a softer pull ring should utilize SS430, which has a higher permeability. Where maximization of sensitivity is not critical (as is the case for larger coils with diameters exceeding 0.150″) or where MRI-compatibility is necessary, an austenitic stainless steel such as SS304 or SS316 may be used for the pull ring body 12. Cobalt or ferrite nanoparticles (not separately shown) can be added or coated onto the pull ring body 12 to increase the magnetic permeability and/or saturation-flux-density. High-strength magnetically active alloys such as Permendur, Vadnadium Permendur, and HiperCo50, which have sufficient hardness for the physical pull ring requirements, can also be used.

The pull ring 12 may be coated with a parylene layer (not separately shown) or other chemically inert dielectric substance to provide electrical insulation and render the pull ring 12 compatible with medical applications. The parylene layer is particularly important in a groove or connection recess 36 where the electrical connections are made to the coil wire leads 26. Alternatively the electrical connections can be made to the coil wire leads 26 using the interconnect ring disclosed in U.S. patent application Ser. No. 16/040,052, incorporated by reference. The parylene or similar layer is also particularly important if nanoparticles or a high-strength magnetically active alloy is used, to reduce the potential for chemical activity.

The coil wire 16 is wound around the pull ring 12, including a plurality of turns so as to be able to sense and/or create an electric, magnetic or electromagnetic field through body (human) tissue as is common in medical imaging. For instance, the coil 16 may be wound with about 100-10000 turns or more around the coil area 28 of the pull ring 12, providing an inductance in the microhenry-millihenry range. The electromagnetic field detector (not shown) is used to sense the position and/or the orientation of the catheter according to the electromagnetic field generated in the vicinity of the catheter. Alternative embodiments use the coil 16 for other purposes, such as for sensing temperature or pressure.

The coil wire 16 is quite thin, typically having a size smaller than 40 AWG, such as within the range of 40-60 AWG. In the preferred examples shown, the coil wire 16 is an insulated 58 AWG copper wire, meaning the copper wire is a tiny thread of about 0.0004 inches in diameter. For comparison, the thickness of a human hair is about 0.002-0.004 inches in diameter, i.e., about five to ten times thicker than the copper conductor of the coil wire 16. Being so very thin, the flexible coil wire 16 is also quite fragile. The coil wire 16 can be closely wound in a single layer around the pull ring 12, but more preferably is closely wound in numerous layers (such as 5 to 20 layers) around the outwardly facing surface of the pull ring 12. Two leads 26 for the coil 16 extend beyond the coil area 28.

A pair of magnet circuit wires 18 are electrically connected to the coil wire leads 26 to carry the signal longitudinally out of the proximal end (not shown) of the catheter. In the preferred embodiment, the circuit wires 18 are substantially larger in diameter than the coil wire, such as with a range from 32 to 46 AWG, provided as a twisted pair within a sheath 30 (drawn somewhat translucent and shorter than its actual length in FIGS. 1 and 5, to better show the twisted pair circuit wires 18) for protective shielding. At this larger diameter, the circuit wires 18 can withstand the twisted pair bending twist as well as the bending of the catheter without breaking, whereas the coil wire, including both the coil 16 and its leads 26, is intended to be entirely stationary relative to the pull ring 12 throughout use of the catheter.

The pull ring 12 itself is preferably formed with one or more geometric features to accommodate the coil 16 and the electrical connections for the coil 16. To accommodate the coil 16 without having the coil 16 stand proud of the outer diameter of the pull ring 12, the coil area 28 of the pull ring 12 is machined to a smaller wall thickness, such as removing 5 to 70% of the full wall thickness. The term “stand proud”, as used herein and relative to full wall thickness, refers to a physical geometry extending outside the shape defined by the full wall thickness if the entire tubular structure of the pull ring had a uniform wall thickness. Thus, since pull ring 12 is cylindrical, the coil 16 does not “stand proud” of the pull ring by having the largest coil turn with an outer diameter which is no greater than the maximum outer diameter of the pull ring 12. For instance, if seven layers of turns of 58 AWG wire are used for the coil 16, the machining can remove about 0.0028″ of material (or slightly less, depending upon how the different coil turn layers are nested into each other) from the outer diameter of the cylindrical tubular pull ring 12. In the example depicted in FIG. 1, this is about 50% of the wall thickness of the pull ring 12.

The coil area 28 is longitudinally in a middle portion of the pull ring 12, between a proximal section 32 of full wall thickness and a distal section 34 of full wall thickness. The two sections 32, 34 of full wall thickness greatly help to maintain the overall shape and rigidity of the pull ring 12, particularly important to avoid damage to the shape during handling of the coil sensor pull ring 10 prior to assembly into a catheter. After assembly into the catheter, the material thickness of the coil area 28 must still withstand the compression and twisting forces seen during catheter deployment and provide sufficient hoop strength to withstand any residual tension in the coil wire 16. (During the winding operation of the coil 16 onto the pull ring 12, the pull ring 12 is supported throughout its inner diameter, so the tension seen during winding does not have to be withstood by the coil area thickness.)

A longitudinally extending connection recess 36 is machined or otherwise formed into the pull ring 12 proximally outside the coil area 28. The connection recess 36 is preferably deep enough such that the circuit wires 18 can be received within the connection recess 36 without standing proud of the outer cylindrical diameter of the pull ring 12.

The circuit wires 18 are electrically connected to the winding wire leads 26 to lead out the coil 16 to a proximal connector (not shown). The electrical connection can be achieved by resistance welding or soldering, with the leads 26 then positioned within the connection recess 36 of the pull ring 12. The termination locations can be protected with heat shrink material (not shown) and/or then potted with adhesive (not shown) to provide a more durable dielectric barrier between the wires 18, 26 and pull ring 12. Such potting provides improved strength to ensure the wires 18, 26 and termination site remain intact during assembly and operation of the catheter.

The preferred connection recess 36 has a planar bottom surface 38. The planar bottom surface 38 of the connection recess 36 provides a flat platform that is better suited for adhering the bond sites via a cyanoacrylate or similar adhesive (not shown). The relatively large surface area of the bottom surface 38 of the connection recess 36 allows a very durable bond. Alternatively, the flat base 38 of the connection recess 36 could provide a platform for adhering bonding-pads or micro printed circuit boards (“PCBs”) (not shown). The flat bonding platform 38 would make such bonding operations more efficient. The preferred machining operation to achieve the flat bottom surface 38 is through milling with an end mill (not shown).

The connection recess 36 improves the quality of the electrical connection and the strength of mechanical connection for the wires 18, 26. The pull strength, particularly on the coil lead wires 26, is improved, resulting in fewer failures. With a better electrical connection, the electrical response of the coil 16 is more accurately transmitted to the circuit wires 18 for reading with appropriate electrical equipment. Manufacturability is improved and made easier, and the resulting EM sensor is more reliable.

FIG. 3 shows a second embodiment of a pull ring 40. In this embodiment, the side walls 42 defining the coil area 44 define planes which are not perpendicular to the pull ring axis 24, but rather are offset or skewed relative to the normal plane of pull ring axis 24 by an offset angle θ. Both side walls 42 define planes which are parallel to each other. Preferred embodiments use an offset angle θ within a range of 1 to 10°, with the most preferred embodiment using an offset angle θ of about 4°. When the coil wire is wound about the pull ring 40, the turns of the coil wire are offset by the same offset angle θ, such as by moving the pull ring 40 longitudinally back and forth (or pivoting the pull ring 40 back and forth) relative to the coil wire source (or vice versa) during each rotation of the pull ring 40 while winding. With the windings of the coil laid off-axis from the pull ring axis 24, the coil can provide compact 6-Degree-of-Freedom tracking capabilities.

FIG. 4 shows a third embodiment of a pull ring 46. This embodiment is similar to FIG. 3, but then adds a second coil area 48 on the pull ring 46. The second coil area 48 is offset relative to the first coil area 44, such as using an offset angle θ₂ of about −4° relative to the normal plane of pull ring axis 24. Separate coils (not shown) are wound in the two distinct coil areas 44, 48, each attached to their own separate circuit wires (not shown) such as within their own separate connection recess 36, 50. This configuration allows more robust 6-Degree-of-Freedom tracking capabilities. Crosstalk between the two coils can be minimized by use of an austenitic stainless material for the pull ring 46, such as SS304 or SS316.

FIG. 5 shows another embodiment of an EM coil sensor pull ring 52. This embodiment 52 shares many of the features of the EM coil sensor pull ring 10 of FIG. 1 and adds a second coil 54 similar to the second coil of FIG. 4, but locates the second coil 54 around one of the pull wires 20. To facilitate better wrapping of the coil 54 around the pull wire 20, at least the portion of the pull wire 20 inside the coil 54 preferably has a circular or ovular or rounded corner cross-sectional shape. For instance, the distal end of a circular cross-sectioned pull wire 20 can be stamped into a rectangular cross-section, to better mate for the laser welding operation into its slot 22 in the pull ring 12. If desired, multiple separate coils (not shown) can be longitudinally spaced along a pull-wire 20, and be used to can provide visualization of the deflection in the catheter shaft. As in the case of the pull ring designs (12 as compared to 40, 46), the windings may be made with an axis parallel to the axis of the underlying pull wire 20 or off-axis to allow 6-DOF localization. However, off-axis winding is much more difficult without machining a side wall (not shown, but similar to side wall 42) into the pull-wire 20. The pull-wire 20 should ideally be spring-tempered and non-magnetic for optimal physical properties and sensor performance. Most preferred material choices for the pull-wire 20 inside the coil 54 include SS304, SS316, and/or nitinol.

The pull-wire 20 should have a cross-sectional area exceeding ≈0.003 in² if a direct-winding approach is to be used. The length of the pull-wire 20 proximal to the coil 54 is generally irrelevant; typical lengths range from 4 to 72″. The winding wire 54 will typically lie between 50 and 58 AWG and be wound over a length of 0.3 to 0.5″. With about eight to twenty layers of windings, the windings 54 add approximately 0.004-0.010″ to the thickness of the pull-wire 20 due to the necessary number of winds.

Other embodiments utilize more than one coil around one pull wire 20, or even use separate coils around each of the pull wires 14, 20, but omit the coil 16 around the pull ring 12. Such embodiments reduce the cost and length of the pull ring 12, potentially decreasing the rigidity of the catheter distal end. For all embodiments which utilize a coil 54 around the pull wire 20 while making connections on a recessed flat 50 of the pull ring, a downside is that the coil 54 can move relative to its leads 26 during flexing of the pull wire 20, which increases the chance of breakage or damage to the thin coil wire 54 and/or its leads 26.

In any of these embodiments, one or more strips (not shown) of higher magnetically permeability material can be added inside the coil wire. The slimness of the strip should not appreciably constrict the working channel of the catheter. Practical magnetic-strip dimensions are as little as 0.001″ to 0.012″ thick and 0.02″ to 0.08″ wide, with a length matching the length of the coil. The sensor coil 16 and/or 54 is wound directly over the magnetic strip as well as around the pull ring or pull wire to which the magnetic strip is attached.

The alloy of the strip is chosen to best fit the application. For many applications where such strip(s) is/are added, the strip(s) should be formed of a traditional high-permeability alloy such as permalloy. For the case of a coil 54 placed over a particularly narrow pull wire (≥0.020″), a high saturation-flux-density alloy such as HiperCo 50 or MetGlass 2605 may be necessary to avoid saturation. For applications where the coil is applied over flexing locations, MetGlass 2714, MetGlass 2605 and similar magnetic glasses have a smaller bending radius than most magnetic alloys, making them ideal for adding the higher permeability strip(s) while maintaining flexibility.

The present invention has at least several primary advantages over prior art solutions. The invention minimizes the intrusion of the EM sensor windings into the working volume of the catheter. Comparable EM sensors in the industry are not wound directly over existing catheter components and require an additional “core” to provide the EM sensor form. The coil sensor pull ring 10 can easily be incorporated into the catheter shaft as a pre-assembled assembly. The invention reduces EM sensor location offset as the coil 16 is automatically ‘centered’ around an existing structure in the catheter, typically having the coil axis 24 coincident with the catheter axis. Winding around a hollow feature, such as a pull ring 12, 40, 46, maintains an ‘open ID’ (open inside diameter—thus having applications for both steerable closed shaft catheters and for steerable introducers used to deliver catheters and medical devices through a central lumen).

Although the present invention has been described with reference to preferred embodiments, workers skilled in the art will recognize that changes may be made in form and detail without departing from the spirit and scope of the invention.

What is claimed is:

1. A microminiature electro-magnetic coil sensor pull ring for use in a medical catheter, comprising:
a tubular pull ring having a full wall thickness, the pull ring having a coil wrap area about a longitudinal axis, the pull ring having a connection recess into the full wall thickness and located proximal to the coil wrap area, the pull ring having at least one pull wire connection location into the full wall thickness and located proximal to the coil wrap area;
a coil formed of a flexible, electrically insulated metal wire wrapped about the coil wrap area with a plurality of turns for sensing through human tissue, the wire being smaller than 40 AWG, the wire terminating in two lead ends extending flexibly from the turns;
at least one pull wire connected to the pull ring within the pull wire connection location, for changing the angle of the pull ring for navigation of the medical catheter through human tissue;
circuit wires electrically connected to the two lead ends within the connection recess, such that neither the circuit wires nor the lead ends stand proud of the full wall thickness.

2. The microminiature electro-magnetic coil sensor pull ring of claim 1, wherein the connection recess has a flat bottom surface.

3. The microminiature electro-magnetic coil sensor pull ring of claim 1, wherein the coil wrap area is recessed relative to the full wall thickness, with a proximal region of full wall thickness on the pull ring proximal to the coil wrap area and with a distal region of full wall thickness on the pull ring distal to the coil wrap area, such that the coil does not stand proud of the full wall thickness.

4. The microminiature electro-magnetic coil sensor pull ring of claim 3, wherein the connection recess extends from a proximal end of the pull ring to the coil wrap area.

5. The microminiature electro-magnetic coil sensor pull ring of claim 3, wherein the coil wrap area is defined by at least one side wall of the pull ring, the side wall defining a plane which has an offset angle relative to a plane normal to an axis of the pull ring, and wherein the turns of the coil wire are offset by the same offset angle.

6. The microminiature electro-magnetic coil sensor pull ring of claim 5, wherein a second coil wrap area is defined by a second side wall of the pull ring, with a second coil wrapped about the second coil wrap area with a plurality of turns for sensing through human tissue, wherein the second side wall has a second offset angle relative to a plane normal to an axis of the pull ring, and wherein the turns of the second coil wire are offset by the second offset angle, such that the turns of the second coil are not parallel to the turns of the coil.

7. The microminiature electro-magnetic coil sensor pull ring of claim 5, wherein the pull ring is formed of an austenitic stainless steel material.

8. The microminiature electro-magnetic coil sensor pull ring of claim 1, further comprising a second coil formed of a flexible, electrically insulated metal wire wrapped about the pull wire with a plurality of turns for sensing through human tissue, the wire being smaller than 40 AWG.

9. The microminiature electro-magnetic coil sensor pull ring of claim 8, where the pull ring has a second connection recess into the full wall thickness and located proximal to the coil wrap area, for connecting circuit wires to leads for the second coil.

10. The microminiature electro-magnetic coil sensor pull ring of claim 1, wherein the pull ring is formed of a 400-series ferritic stainless steel material.

11. The microminiature electro-magnetic coil sensor pull ring of claim 1, wherein the circuit wires are with a range from 32 to 46 AWG, provided as a twisted pair within a sheath.

12. The microminiature electro-magnetic coil sensor pull ring of claim 1, wherein the coil is wound with 100 to 10000 turns in 5 to 20 layers.

13. A method of manufacturing a microminiature electro-magnetic coil sensor pull ring for use in a medical catheter, comprising:
forming a tubular pull ring, the tubular pull ring having a full wall thickness about a longitudinal axis;
machining a coil wrap area by removing material from an outer side of the full wall thickness;
machining an electrical connection recess into the full wall thickness and located proximal to the coil wrap area;
machining at least one pull wire connection location into the full wall thickness and located proximal to the coil wrap area;
winding a coil about the coil wrap area, the coil being formed of a flexible, electrically insulated metal wire with a plurality of turns for sensing through human tissue, the wire being smaller than 40 AWG, the wire terminating in two lead ends extending flexibly from the turns;
attaching at least one pull wire to the pull ring within the pull wire connection location, the pull wire being adapted for changing the angle of the pull ring for navigation of the medical catheter through human tissue; and
electrically connecting circuit wires to the two lead ends within the connection recess, such that neither the circuit wires nor the lead ends stand proud of the full wall thickness.

14. The method of claim 13, wherein the electrical connection recess is machined to provide a flat bottom surface.

15. The method of claim 13, wherein the coil wrap area is machined between a proximal region of full wall thickness on the pull ring proximal to the coil wrap area and a distal region of full wall thickness on the pull ring distal to the coil wrap area, wherein the coil is wound such that the coil does not stand proud of the full wall thickness, and wherein the connection recess is machined to extend from a proximal end of the pull ring to the coil wrap area.

16. The method of claim 13, wherein the pull wire has a circular cross-section, and further comprising:
stamping a distal end of the circular cross-sectioned pull wire into a rectangular cross-section prior to the attaching act.

17. A microminiature electro-magnetic coil sensor pull ring for use in a medical catheter, comprising:
a tubular pull ring having at least one pull wire connection location;
at least one pull wire connected to the pull ring at the pull wire connection location, for changing the angle of the pull ring for navigation of the medical catheter through human tissue;
a coil formed of a flexible, electrically insulated metal wire wrapped about the pull wire with a plurality of turns for sensing through human tissue, the wire being smaller than 40 AWG.

18. The microminiature electro-magnetic coil sensor pull ring of claim 17, wherein the coil wire terminates in two lead ends extending flexibly from the turns; wherein the pull ring has a connection recess relative to a full wall thickness of the tubular pull ring, and further comprising:
circuit wires electrically connected to the two lead ends within the connection recess, such that neither the circuit wires nor the lead ends stand proud of the full wall thickness.

19. The microminiature electro-magnetic coil sensor pull ring of claim 18, wherein the pull ring has a coil wrap area recessed relative to the full wall thickness, with a proximal region of full wall thickness on the pull ring proximal to the coil wrap area and with a distal region of full wall thickness on the pull ring distal to the coil wrap area, and further comprising:
a second coil wound about the coil wrap area of the pull ring.

20. The microminiature electro-magnetic coil sensor pull ring of claim 19, wherein the second coil does not stand proud of the full wall thickness.
